(19) United States
(12) Patent Application Publication
(10) Pub. No.: US 2014/0249382 A1
(43) Pub. Date: Sep. 4, 2014

(54) MOTION DETECTION SYSTEM

(71) Applicants: Nikhil BHAT, Fremont, CA (US); George Y. CHOI, Atherton, CA (US)

(72) Inventors: Nikhil BHAT, Fremont, CA (US); George Y. CHOI, Atherton, CA (US)

(21) Appl. No.: 14/276,283

(22) Filed: May 13, 2014

Related U.S. Application Data
(63) Continuation of application No. 12/537,540, filed on Aug. 7, 2009.

(51) Int. Cl. A61B 5/00

(52) U.S. Cl. CPC A61B 5/4818
USPC 600/301; 600/595; 600/407

(57) ABSTRACT

A motion detection system for detecting movement and/or breathing from a subject, e.g., for preventing SIDS, can incorporate a sensor in communication with a fluid-filled chamber while optionally combining audio and/or video monitoring systems. The sensor module may have a housing which can be attached, e.g., to an article of clothing worn by the subject. The sensor may be entirely enclosed within the housing which may also be filled with a vibrationally transmissive medium which transmits movements from the subject such that these signals impinge upon the sensor which captures these signals (or the absence of these signals) for processing and possibly alerting a parent or caretaker.

### CROSS-REFERENCE TO RELATED APPLICATIONS

This application is a continuation of U.S. patent application Ser. No. 12/537,540 filed Aug. 7, 2009, which is incorporated herein by reference in its entirety.

### FIELD OF THE INVENTION

The present invention relates to devices and methods for detecting the motion of a subject. More particularly, the present invention relates to devices and methods for detecting and/or monitoring the motion of a subject, such as an infant.

### BACKGROUND OF THE INVENTION

Sudden Infant Death Syndrome (SIDS) typically occurs in infants under one year of age where the infant may stop breathing and cease movement. In many cases, parents or caregivers typically await for the movement or motion of the infant as an indicator that the infant is merely sleeping and not in distress.

SIDS may occur in healthy infants as a result of simultaneous occurrence of a series of biological events such as periods of apnea, difficulty in breathing, changes in muscle tone, etc. These episodes may occur during sleep, feeding, or while awake and are potentially life-threatening events.

Previous devices have utilized breathing monitors and alarm systems to monitor the breathing of infants. These monitors have typically utilized electrodes attached to the skin of the infant. Other types of SIDS monitoring equipment have utilized various mechanisms such as accelerometers. However, these home monitoring systems are subject to false alarms making monitoring of an infant difficult. Video monitoring equipment is widely utilized but they are relatively expensive and fail to adequately convey information when an infant is quite and not moving. Additionally, audio monitoring equipment likewise fails to transmit adequate information and can confusingly transmit undesirable background noise.

Other types of devices have included pulse oximeter devices for monitoring the oxygen saturation levels in the infant. Unfortunately, such devices particularly when used with accelerometer-type devices are still subject to many false alarms. Other types of monitoring systems have proven to be overly complex and expensive.

Thus, a relatively inexpensive monitoring system which can be readily applied to an infant (or adult) for detecting movement (or lack of movement) and/or breathing is desirable.

### BRIEF SUMMARY OF THE INVENTION

A system for detecting motion and/or breathing from a subject, e.g., for preventing SIDS, can be configured in a number of different ways while optionally combining audio and/or video monitoring systems. Such a system may incorporate a motion and/or breathing sensor module which may comprise a sensor in communication with a fluid-filled chamber. The sensor itself may be in contact with the fluid-filled chamber or alternatively it may be partially or completely enclosed within the chamber. The sensor module may have a housing to which one or more connectors may be attached for coupling or connecting, e.g., to an article of clothing worn by the subject. The sensor may be entirely enclosed within the housing which may also be filled with a vibrationally transmissive medium such as a fluid or gas, e.g., water (such as de-ionized water), saline, air, gel, etc.

While the sensor may be positioned within the fluid-filled housing in a cantilevered configuration where a single end of the sensor is securely coupled or attached within the housing, the sensor may be positioned within the fluid-filled housing in various locations and configurations.

An electronics assembly, e.g., a circuit board, may also be integrated within or upon the housing such that the assembly is in electrical communication with the sensor. The electronics assembly may provide for various functions of the signals detected by the sensor. For instance, the assembly may be configured to amplify the detected signals as well as provide for filtering of the signals as well as various other functions.

The housing may also have a contact surface for positioning against the subject's body. Thus, the contact surface may be optionally configured to be made of a soft and/or flexible material such as silicone, polyurethane, etc. which is also transmissive of movements and/or vibrations from the subject through the surface and into the housing. The other portions of the housing may be fabricated similarly or made from any variety of materials such as various plastics and/or metals.

In use, with the sensor module positioned against or in proximity to the subject, any movements or motion such as the movement of a limb or the movement the subject's chest or abdomen resulting from respiration, may be transmitted through the contact surface and into the housing. The movements or motion may then be captured by the fluid-filled chamber and vibrationally transmitted through the transmissive medium such that these signals impinge upon the sensor which may then capture these signals (or the absence of these signals) for processing.

Generally, the sensor may comprises a piezoelectric film sensor but other sensors such as electroactive polymers including ionic polymer metal composite (IPMC) sensors, piezoelectric sensor strain gauges, variable resistance sensors, etc., can also be used. Typically, IPMC sensors are ionic electroactive polymers which may comprise an ion exchange membrane such as Nafion or Flemion which may be plated on either side with a conductive material, e.g., platinum, gold, etc. Alternative electroactive polymer materials which may also be utilized for the sensor may also include, e.g., conducting polymers such as polypyrrole, polyaniline, polythiophene, polyacetylene, carbon nanotube based sensors, ionic gels, dielectric elastomers, etc. Various shapes for the sensor which are practicable may be utilized.

The circuit board itself may generally comprise a printed circuit board having a sensor signal conditioning circuit which may clean or filter the sensor signal received from the sensor through the conductor. The cleaned or filtered sensor signals may then be transmitted through the conductor to a sensor signal amplifier which may amplify the sensor signal, if necessary. The microcontroller may be electrically coupled to the amplifier via conductor and may continuously monitor the signal received. The microcontroller may have a preprogrammed algorithm which may take in the sensor signal and based on the signal, indicate a particular status of the monitored subject. A wireless transmitter in communication with the microcontroller may also be located on the circuit board for transmitting the processed information from the microcontroller to a remote receiver. The wireless transmitter may transmit the information either continuously based on the sampling rate and transmission frequency or intermittently. Alternatively, the transmitter may transmit the information only when a fault condition is detected to alert the caregiver monitoring the subject.

In an example of monitoring an infant to determine whether breathing has ceased, once the system has been started or initiated the sensor module may be set to await the detection of any vibrations through the transmissive medium. Movement or motion from the infant's chest or abdomen normally moving during respiration (or other typical body movements and even including vibrations from the infant's beating heart) may transmit vibrations through the fluid-filled chamber via the transmissive medium where the sensor may receive these transmitted vibrations. Thus, if or when vibrations are detected by the sensor such as when a breathing event may create ripples in the fluid or gas and when these vibrations impinge upon the sensor, the sensor may output an electrical signal. Because the sensitivity of the sensor may be varied or adjusted (e.g., preset or adjusted by the parent or caretaker), the output of the electrical signal may accordingly vary depending upon the adjustment.

For every movement or breathing event by the monitored subject, the sensor will output a value which may be compared against a nominal threshold signal value. Thus, if the sensor output is greater than or equal to the programmed threshold signal value, this will indicate to the microcontroller that a movement or breathing event is occurring and the system may continue to monitor the subject. However, if the sensor output is less than the programmed threshold signal value, then the microcontroller may be programmed to signal an alarm to a third party, such as the parent or caretaker, that the monitored subject has ceased movement or breathing.

Typically, an infant is estimated to take about 30 to 50 breaths/minute. If no movement or breathing is detected in the infant for a predetermined period of time by the sensor module, for example, 10 sec, 15 sec, 20 sec, or any other minimum time period preset and/or set by the parent or caretaker, the microcontroller may automatically signal an alarm to alert the parent or caretaker of this cessation of movement and/or breathing.

In positioning or placing the sensor modules over, upon, or in proximity to the monitored subject, a single sensor module may be positioned for instance upon the subject's chest or abdomen to detect the subject's movements associated with respiration, e.g., as the subject inhales and/or exhales, the sensor module may detect the associated movement of the chest or abdomen. Yet in other examples, multiple sensor modules may be used in combination with one another and positioned along various regions upon or in proximity to the subject's body. In one example, a central monitoring system (which may be monitored by the parent or caretaker) may be in communication with multiple sensor modules. In this example, a first module may be positioned, e.g., on the belly or abdomen, a second module may be positioned, e.g., on the lower back, a third module may be positioned, e.g., on the chest, while a fourth module may be positioned, e.g., on the upper back. Each of the sensor modules may be integrated along an article of clothing such as a diaper or a bodysuit within pockets or they may be positioned via one or more optional straps which are flexible and may be integrated within or along the bodysuit or worn separately from the bodysuit. Although four separate sensor modules are illustrated, a single sensor module may be used and selectively positioned upon the infant or along an article of clothing worn by the infant. Alternatively, more than four sensor modules may be used over various regions of the infant, if so desired or necessary.

In addition to sensing the motion and/or breathing of the monitored subject, the monitoring system may further include a number of additional features such as audio and/or video monitoring, each of which may be used individually or in combination with one another. In this example, the monitoring system may include at least one sensor module in contact or in proximity to the monitored subject, e.g., infant. The at least one sensor module may be in electrical communication, e.g., wireless or wired, with the stationary transmitter which may be positioned in proximity to the monitored subject. Wireless transmission between the sensor module and transmitter may be based on the any number of wireless transmission protocols, such as ZIGBEE® (Zigbee Alliance, San Ramon, Calif.) or any other similar data transmission protocol such as BLUETOOTH® (Bluetooth Sig., Inc., Bellevue, Wash.) which allow for wireless communication in ranges up to 100 feet or more from the receiver unit. The stationary transmitter may optionally include an audio module (including, e.g., a microphone) for detecting any auditory information from the subject, such as crying, movement, auditory signs of respiration, etc. The transmitter may also include an amplifier as well as an amplifier gain control for adjusting the amplifier gain. A power button may also be incorporated to separately power on or off the transmitter if so desired while the transmitter may be powered by battery or through a standard outlet.

Aside from the sensor module and optional audio module, an additional video camera may also be included for visually monitoring the subject. The video camera may be in communication, e.g., wireless or wired, with the receiver unit as well such that the parent or caretaker may also optionally visually monitor the subject. Wireless video communication may likewise be based on the any number of wireless transmission protocols as described above. The video camera may be accordingly located in proximity to the monitored subject, e.g., mounted on a wall or on a crib or stand to capture video images of the subject. The video camera may also incorporate an audio module for also capturing auditory information from the subject. As the video camera may also incorporate a wireless or wired transmitter and/or receiver to transmit or receive signals from the sensor module and/or audio module as well as the transmitter, the camera may also include a microcontroller or video processor to integrate the sensor signal along with the video and/or audio signal as well. As the video camera provides visual images of the monitored subject, the receiver unit may accordingly incorporate a visual display, such as an LCD display, to show the video and movement and/or breathing patterns from the subject, as described in further detail below. Additionally, the receiver unit may also incorporate a microcontroller to set an alarm threshold as well, as also described in detail herein.

In another variation, a sleep positioner comprising a bedding having one or two adjustable protrusions or obstructions, e.g., side wedges, which protrude from the bedding may also be used. One or more sensor modules may be enclosed within or protrude from one or both wedges such that the infant is in contact with at least one sensor module when positioned upon the bedding. The bedding, which may be used in combination with the wedges or alone, may also be configured to include one or more sensor modules in contact with a fluid filled chamber. Such a bedding may include a base layer, a second fluid or gas filled layer laid atop the base layer, and a third bedding layer laid atop the fluid or gas filled layer. The fluid or gas filled layer may transmit movement of the monitored subject through the fluid or gas as vibrations to an integrated sensor module, which may be integrated within the base layer or fluid or gas filled layer. The vibrations sensed by the sensor module may be detected and transmitted via a communication cable to a receiver or other external unit.

### BRIEF DESCRIPTION OF THE DRAWINGS

FIG. 1A illustrates a perspective transparent view of one example of a sensor module having a sensor positioned within and which may be readily positioned against or in proximity to a subject.

FIGS. 1B and 1C illustrate various perspective views of the sensor module of FIG. 1A.

FIGS. 2A and 2B illustrate perspective views of a variation of the sensor positioned within the module.

FIGS. 3A to 3D illustrate top views of examples of other alternative sensor configurations.

FIG. 4A schematically illustrates one variation of the layout for a circuit board which may be utilized for processing sensed motion detection signals.

FIG. 4B illustrates a flowchart of one example of an algorithm for determining whether the monitored subject is moving and/or breathing.

FIG. 5 illustrates a partial cross-sectional side view of another variation of a sensor module having a flexible housing.

FIG. 6 illustrates a partial cross-sectional side view of another variation of a sensor module utilizing a biased chamber for transmitting motion to the sensor within.

FIG. 7 illustrates a partial cross-sectional side view of another variation of a sensor module having a sensor supported by one or more flexible membranes or members.

FIG. 8 illustrates a partial cross-sectional side view of another variation of a sensor module which may be actuated by a pivoting or rotating member for transmitting motion.

FIG. 9 illustrates a partial cross-sectional side view of another variation of a sensor module having one or more mass elements positioned within for transmitting motion to the sensor.

FIG. 10 illustrates a partial cross-sectional side view of another variation of a sensor module having two or more sensors positioned within.

FIG. 11 illustrates a partial cross-sectional side view of another variation of a sensor module having two or more sensors positioned within and a mass element suspended between the sensors.

FIG. 12A schematically illustrates an example of a system utilizing multiple sensor modules which may be positioned at various locations in proximity to and/or around a subject.

FIGS. 12B and 12C illustrate exemplary locations for placement of one or more sensor modules upon an article of clothing such as an infant bodysuit.

FIG. 13 schematically illustrates another example of a system utilized in combination with a video module and a mobile receiver.

FIG. 14 illustrates a respiration pattern of an infant detected with a sensor module.

FIG. 15 illustrates an example of a sensor module which may be attached to an article of clothing such as a diaper.

FIG. 16 illustrates a perspective view of an example of an infant sleep positioner which may incorporate one or more sensor modules.

FIGS. 17A and 17B illustrate transparent perspective and end views, respectively, of a sleep positioner incorporating one or more sensor modules in at least one side wedge.

FIGS. 18A and 18B illustrate transparent perspective and end views, respectively, of a sleep positioner incorporating one or more sensor modules extending from at least one side wedge for direct contact against an infant.

FIGS. 19A and 19B illustrate various perspective and transparent perspective views, respectively, of a bedding system incorporating at least one sensor module.

FIGS. 20A and 20B illustrate perspective views of the bedding system showing an example of a layer incorporating fluid channels for transmitting detected motion to the sensor module.

FIG. 21 illustrates a perspective assembly view of an example of how the sensor module may be incorporated with a video module and a central module.

FIGS. 22A and 22B illustrate examples of how the various modules may communication and/or transmit information between one another, e.g., wirelessly.

### DETAILED DESCRIPTION OF THE INVENTION

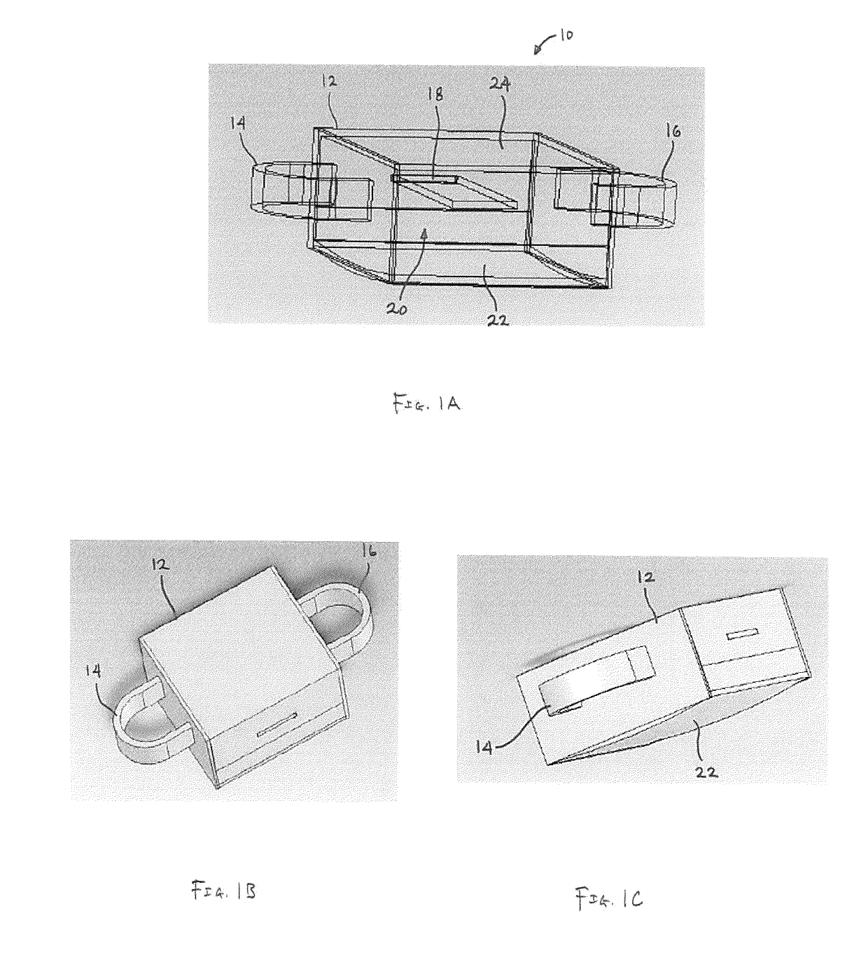

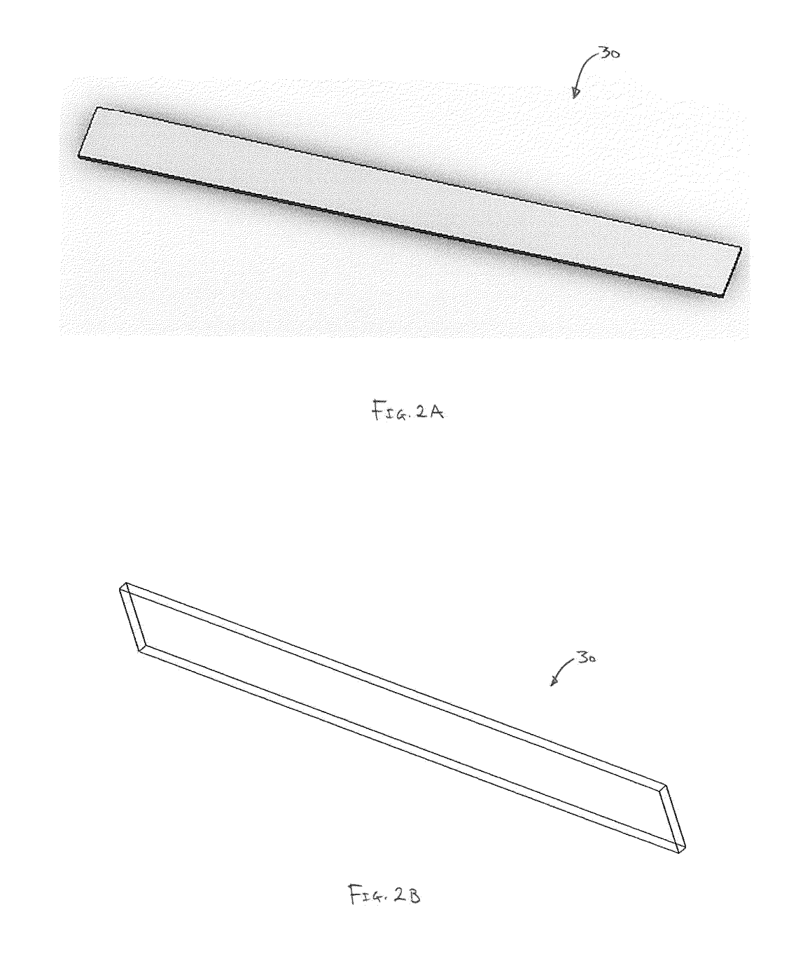

A low cost and low power monitoring system for detecting motion and/or breathing from a subject can be configured in a number of different ways while optionally combining audio and/or video monitoring systems. Generally, such a system may incorporate a motion and/or breathing sensor module which may comprise a sensor in communication with a fluid-filled chamber. The sensor itself may be in contact with the fluid-filled chamber or alternatively it may be partially or completely enclosed within the chamber. As shown in the transparent perspective view of FIG. 1A and the perspective views of FIGS. 1B and IC, sensor module 10 is illustrated as having a housing 12 to which one or more connectors 14, 16 may be attached for coupling or connecting, e.g., to an article of clothing worn by the subject. In this variation, sensor 18 is illustrated as being entirely enclosed within housing 12 which may also be filled with a vibrationally transmissive medium 20 such as a fluid or gas, e.g., water (such as de-ionized water), saline, air, gel, etc.

While sensor 18 is illustrated as being positioned within the fluid-filled housing 12 in a cantilevered configuration where a single end of sensor 18 is securely coupled or attached within housing 12, sensor 18 may be positioned within the fluid-filled housing in various locations and configurations. In other variations, rather than having sensor 18 directly surrounded by the transmissive medium 20, sensor 18 may be external to the fluid chamber and/or to the housing and merely in contact with the fluid chamber. Alternatively, sensor 18 may be partially surrounded by the transmissive medium 20.

An electronics assembly 24, e.g., a circuit board, may also be integrated within or upon housing 12 such that assembly 24 is in electrical communication with sensor 18. Electronics assembly 24 may provide for various functions of the signals detected by sensor 18. For instance, assembly 24 may be configured to amplify the detected signals as well as provide for filtering of the signals as well as various other functions, as described in further detail below.

Housing 12 may also have a contact surface 22 for positioning against the subject's body. Thus, contact surface 22 may be optionally configured to be made of a soft and/or flexible material such as silicone, polyurethane, etc. which is also transmissive of movements and/or vibrations from the subject through the surface 22 and into housing 12. The other portions of the housing 12 may be fabricated similarly or made from any variety of materials such as various plastics and/or metals.

In use, with the sensor module 10 positioned against or in proximity to the subject, any movements or motion such as the movement of a limb or the movement of the subject's chest or abdomen resulting from respiration or vibrations through the chest from the beating heart, may be transmitted through contact surface 22 and into housing 12. The movements or motion may then be captured by the fluid-filled chamber and vibrationally transmitted through the transmissive medium 20 such that these signals impinge upon the sensor 18 which may then capture these signals (or the absence of these signals) for processing.

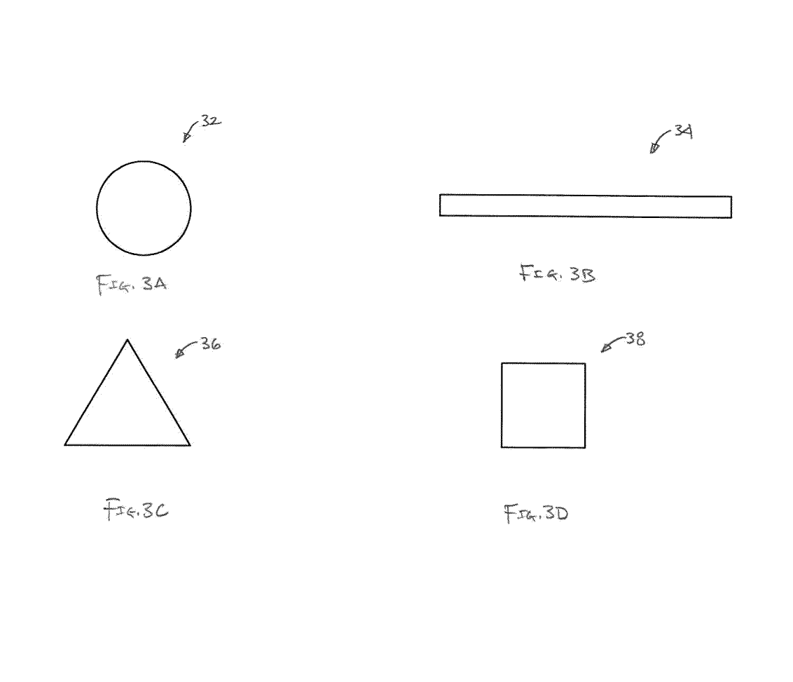

FIGS. 2A and 2B illustrate perspective views of one example of sensor 30 which is configured as an elongate element. Sensor 30 may range in size from, e.g., 0.5 mm to 100 mm in length, 0.5 to 100 mm in width, and 0.01 mm to 10 mm in thickness. Generally, sensor 30 may comprises a piezoelectric film sensor but other sensors such as electroactive polymers including ionic polymer metal composite (IPMC) sensors, piezoelectric sensor strain gauges, variable resistance sensors, etc., can also be used. Typically, IPMC sensors are ionic electroactive polymers which may comprise an ion exchange membrane such as Nafion or Flemion which may be plated on either side with a conductive material, e.g., platinum, uold, etc. Alternative electroactive polymer materials which may also be utilized for sensor 30 may also include, e.g., conducting polymers such as polypyrrole, polyaniline, polythiophene, polyacetylene, carbon nanotube based sensors, ionic gels, dielectric elastomers, etc.

Such IPMC sensors can be made to work in both wet and dry environments. In the case of sensor 30 being positioned in a dry environment within housing 12, such as where a gas may be used as the transmissive medium, sensor 30 may be soaked in a ionic liquid (e.g., liquids comprised predominantly of ions and ion-pairs at some given temperature) and then coated with parylene or other similar material. In the case of sensor 30 being positioned in a wet environment within housing 12, such as where a fluid like saline is used as the transmissive medium, sensor 30 may be configured into its sodium form to operate within such a saline environment. In either case, the sensitivity of sensor 30 may be optionally varied and/or adjustable to suit different environments and/or operating conditions. Optionally, piezoelectric film sensors can also be covered with a fluid-impermeable coating such as parylene, silicone, polyurethane, etc., for use when the sensor is either partially or fully submerged within the transmissive medium.

Aside from an elongate rectangular shape, sensor 30 may be alternatively configured into a variety of shapes. For example, FIG. 3A shows a top view of an alternative sensor 32 configured into a circular shape; FIG. 3B shows a top view of another sensor 34 configured into an elongated rectangular shape; FIG. 3C illustrates a top view of another sensor 36 configured into a triangular shape; and FIG. 3D illustrates a top view of yet another sensor 38 configured into a square shape. These figures are merely exemplary of the various sensor configurations and are not intended to be limiting. Other shapes which are practicable may be alternatively utilized.

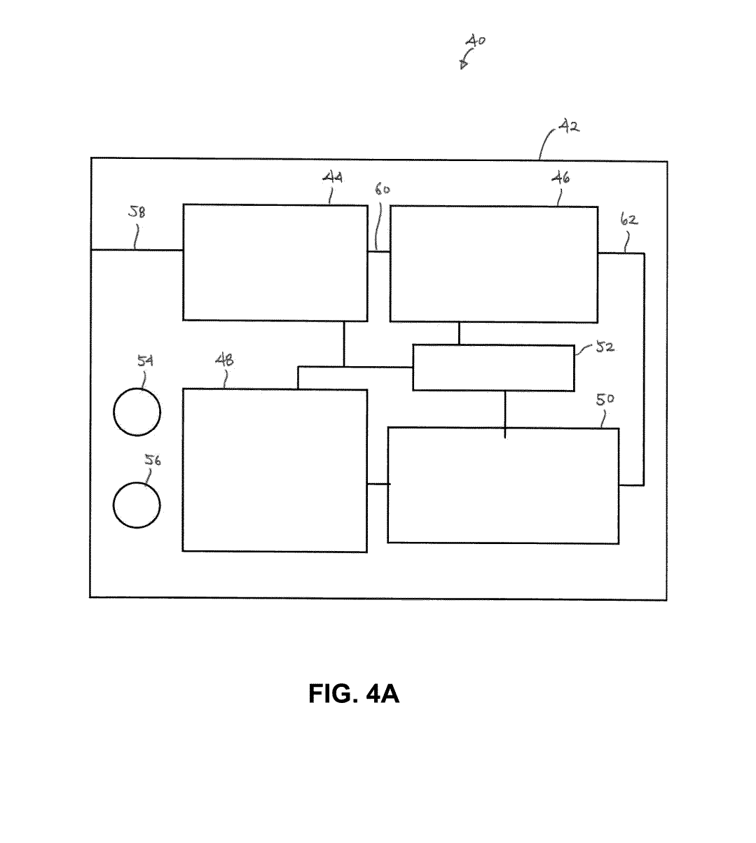

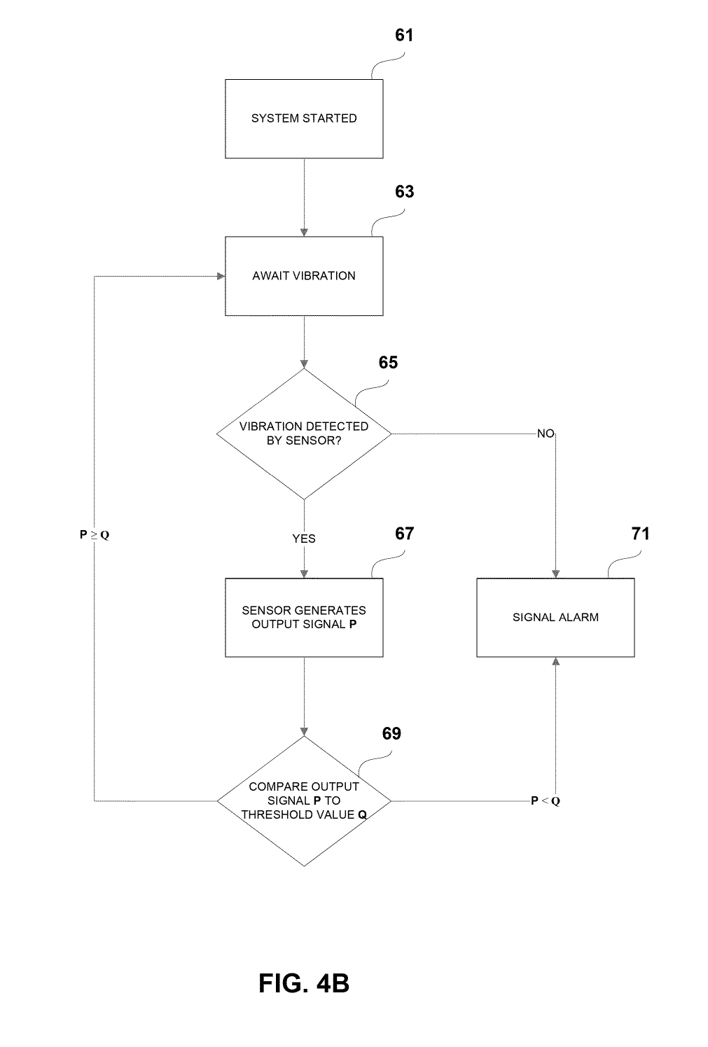

As previously mentioned, the on-board electronics assembly integrated within or along the housing 12 of the sensor module may be programmed to provide any number of functions. As illustrated in the schematic layout of FIG. 4A, one example is shown of circuit board 40 which may be in electrical communication with the sensor positioned within the housing 12. As shown, circuit board 40 may generally comprise a printed circuit board 42 having a sensor signal conditioning circuit 44 which may clean or filter the sensor signal received from the sensor through conductor 58. The cleaned or filtered sensor signals may then be transmitted through conductor 60 to a sensor signal amplifier 46 which may amplify the sensor signal, if necessary. The microcontroller 50 may be electrically coupled to amplifier 46 via conductor 62 and may continuously monitor the signal received.

Microcontroller 50 may have a preprogrammed algorithm which may take in the sensor signal and based on the signal, indicate a particular status of the monitored subject. A wireless transmitter 48 in communication with the microcontroller 50 may also be located on the circuit board 40 for transmitting the processed information from the microcontroller 50 to a remote receiver (as described in further detail below). The wireless transmitter 48 may transmit the information either continuously based on the sampling rate and transmission frequency or intermittently. Alternatively, transmitter 48 may transmit the information only when a fault condition is detected to alert the caregiver monitoring the subject.

Circuit board 40 may also include a rechargeable, permanent, or replaceable power supply 52, e.g., battery, to provide power to each of the individual components. Because the power usage of the system may be relatively low, the power consumption of the entire system may allow for a lengthened life of the power supply 52, e.g., at least one year or longer. Moreover, a power switch 54 may also be included for switching the power off when not in use. Additionally, an audible and/or visual alarm 56, e.g., LED, may also be optionally included to indicate the status of the system or indicate any cessation of motion in the monitored subject, e.g., stoppage in breathing movements.

An example of an algorithm for programming the microcontroller 50 for use with the sensor 18 within housing 12 may utilize the movements and/or motion of the monitored subject or the lack of movement from the subject. In the example of monitoring an infant to determine whether breathing has ceased, as shown in FIG. 4B, once the system has been started or initiated, as indicated in step 61, the sensor module may be set to await the detection of any vibrations through the transmissive medium, as indicated in step 63. Movement or motion from the infant's chest or abdomen normally moving during respiration (or other typical body movements and even including vibrations from the infant's beating heart) may transmit vibrations through the fluid-filled chamber via the transmissive medium 20 where the sensor 18 may receive these transmitted vibrations. Thus, if or when vibrations are detected, indicated in step 65, by the sensor 18 such as when a breathing event may create ripples in the fluid or gas 20 and when these vibrations impinge upon sensor 18, the sensor 18 may output an electrical signal P, as indicated in step 67. Because the sensitivity of the sensor 18 may be varied or adjusted (e.g., preset or adjusted by the parent or caretaker), the output of electrical signal P may accordingly vary depending upon the adjustment.

For every movement or breathing event by the monitored subject, the sensor 18 will output a value P. A nominal threshold signal value of Q may be programmed into microcontroller 50 to indicate a threshold movement or breathing rate. Thus, if the sensor output P is greater than or equal to the programmed threshold signal value Q, this will indicate to microcontroller that a movement or breathing event is occurring and the system may continue to monitor the subject, as indicated in step 69. However, if the sensor output P is less than the programmed threshold signal value Q, then microcontroller may be programmed to signal an alarm to a third party, such as the parent or caretaker, that the monitored subject has ceased movement or breathing, as indicated in step 71. Microcontroller may update an internal counter R for every detected breathing event P.

Typically, an infant is estimated to take about 30 to 50 breaths/minute. If no movement or breathing is detected in the infant for a predetermined period of time by the sensor module, for example, 10 sec, 15 sec, 20 sec, or any other minimum time period preset and/or set by the parent or caretaker, the microcontroller 50 may automatically signal an alarm to alert the parent or caretaker of this cessation of movement and/or breathing, as indicated in steps 65 and 71.

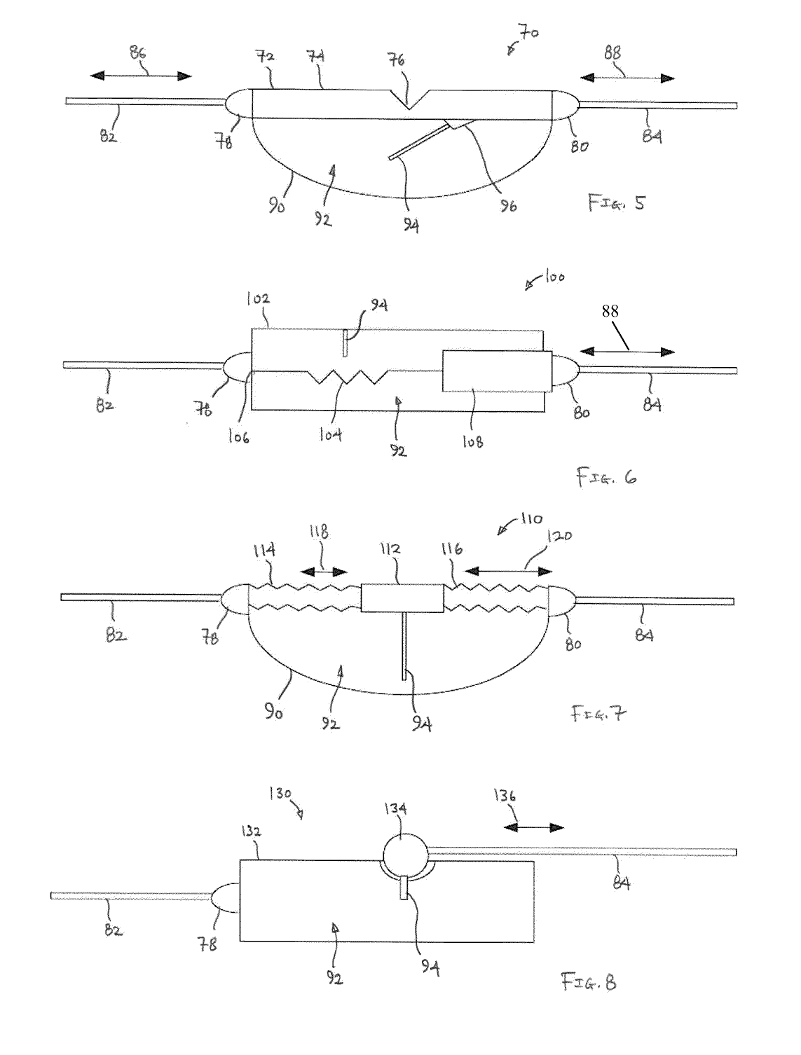

The sensor module itself may be configured in a manner as previously described or in any number of alternative configurations. Another example is illustrated in the partially transparent side view of FIG. 5, which shows a sensor module 70 having a housing 72 which may have an integrated circuit board 74. In this variation, housing 72 may also comprise a hinge or joint 76, e.g., living hinge, to provide housing 72 with additional flexibility for additional comfort when placed against the monitored subject. Housing 72 may also have one or more connectors 78, 80 for attachment to an article of clothing or directly to the monitored subject via one or more coupling members 82, 84, e.g., belts, straps, etc. The transmissive medium 92 may be contained within a chamber surrounded by a contact surface 90 which may be fabricated from a soft, flexible material such as silicone, polyurethane, etc. for direct contact against the monitored subject or in contact with clothing of the subject or in proximity to the subject.

Sensor 94 may be positioned within the fluid-filled chamber connected to housing 72 in a cantilevered configuration via attachment 96 while surrounded by the transmissive medium 92. Sensor 94 may be cantilevered within the fluid chamber while positioned at any number of angles relative to housing 72. In this variation, contact surface 90 may be placed against the monitored subject with coupling members 82, 84 maintaining a position of sensor module 70 relative to the subject. As the subject moves or breathes normally, one or both of the coupling members 82, 84 may move in accordance, as indicated respectively by the direction of movement 86, 88, such that the resulting movements are transmitted as vibrations through housing 72 and/or transmissive medium 92. Additionally and/or alternatively, movement and/or breathing motions may also be transmitted directly through contact surface 90 and through transmissive medium 92 for detection by sensor 94.

FIG. 6 illustrates another variation in sensor module 100 which may comprise a dampener 108 having a compressive chamber with a piston-like member within coupled to a first end of a biasing member 104, e.g., a spring, contained within housing 102 while surrounded by transmissive medium 92. A second end of biasing member 104 may be attached within housing 102 via attachment 106 and sensor 94 may also be positioned within housing 102 while surrounded by medium 92. As one or both of the coupling members 82, 84 are moved in accordance with the subject's movements, dampener 108 (or the piston contained within dampener 108) may be translated relative to housing 102 while biasing member 104 forces dampener 108 to oscillate back into position. These oscillations may be transmitted through medium 92 and to ultimately impinge upon sensor 94, which may then detect the motions accordingly.

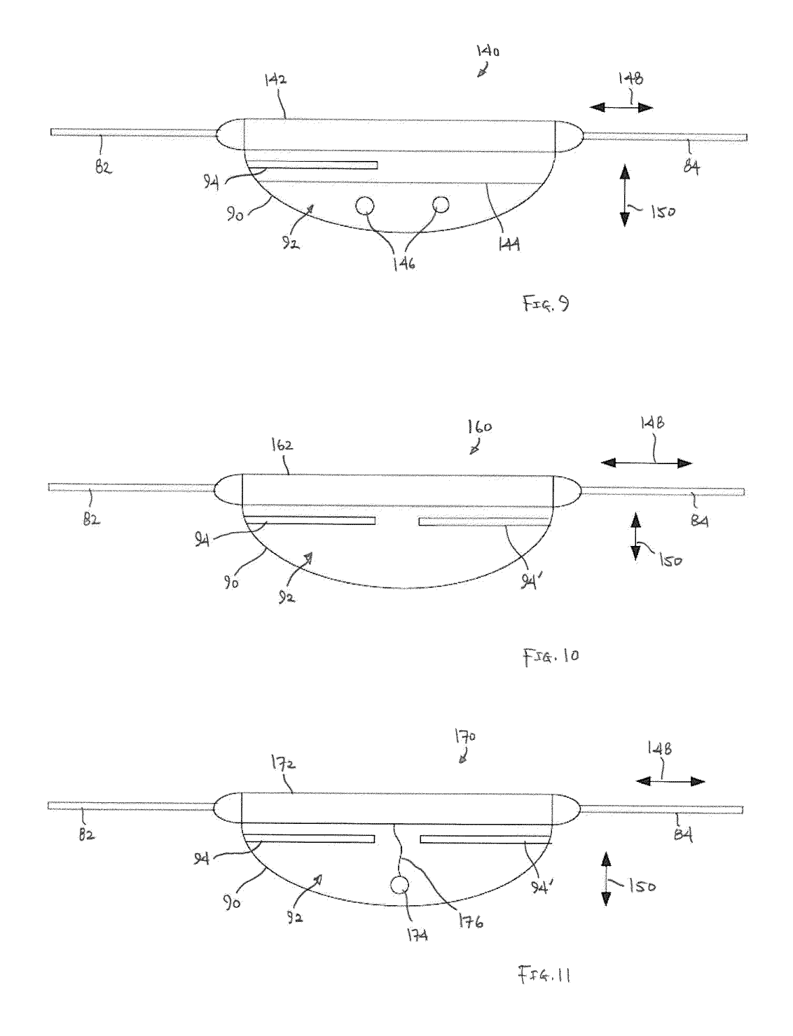

In yet another variation, FIG. 7 shows a sensor module 110 where a platform 112 having sensor 94 attached and extending therefrom within medium 92 may be suspended between one or more members 114, 116 of elastic or distensible material. As the monitored subject moves and/or breathes, such movement may be transmitted through one or both members 114, 116, as indicated by the direction of motion 118, 120, and/or through contact surface 90 such that the vibrations travel through medium 92 and impinge upon sensor 94 for detection.

FIG. 8 shows another variation where sensor module 130 may comprise a housing 132 having sensor 94 positioned within surrounded by medium 92. Sensor 94 may be attached to a pivot or joint 134, e.g., ball-and-socket joint, hinge, etc., which is also coupled to one or more coupling members 82, 84. In this example, as coupling member 84 is tensioned by the subject movement, as indicated by direction of motion 136, pivot or joint 134 may rock or rotate relative to housing 132 such that sensor 94 is also forced to rotate or angle within housing 132 such that rotation of sensor 94 within medium 92 causes sensor 94 to detect the movement accordingly.

Another variation is shown in FIG. 9 of sensor module 140. In this example, housing 142 and contact surface 90 may enclose sensor 94 positioned within in a cantilevered configuration parallel to housing 142. Although illustrated in a parallel configuration, sensor 94 may be angled in a variety of different configurations. A fluid permeable membrane 144, e.g., mesh, may be suspended within the medium 92 such that the fluid chamber is separated into at least two compartments, one with sensor 94 and the remaining one with one or more mass elements 146, e.g., balls, etc., appropriately sized and free-floating within. As sensor module 140 is subjected to various movements by the subject, as indicated by the direction of motion 148, 150, the one or more mass elements 146 may be oscillated or moved within medium 92, while prevented from directly contacting sensor 94 by mesh 144, such that the resulting vibrations from the movement of elements 146 are amplified and transmitted through medium 92 and through mesh 144 and against sensor 94 for detection. The mass elements 146 may be fabricated from any number of materials such as plastics, metals, etc.

FIG. 10 shows yet another variation in sensor module 160 in which housing 162 and contact surface 90 may enclose two or more sensors 94, 94′ surrounded by medium 92. In the variation shown, sensors 94, 94′ may each be positioned in a cantilevered configuration in apposition relative to one another although any number of other configurations may also be utilized. With the additional sensor 94′, movement 148, 150 from the monitored subject may be transmitted through medium 92 with sensor module 160 becoming potentially more sensitive to vibrations.

Another variation is shown in FIG. 11, which illustrates sensor module 170 similarly having two or more sensors 94, 94′ enclosed by housing 172 and contact surface 90. A mass element 174, similar to mass elements 146 as previously described, may be suspended by, e.g., a string 176, which extends from housing 172 between sensors 94, 94′ such that mass element 174 is enclosed by contact surface 90 and is free to move within medium 92 while restrained in movement by string 176. As the subject's movement is transmitted to sensor module 170, mass element 174 may oscillate or move within medium 92 while restrained by string 176 such that vibrations are generated thereby and transmitted through medium 92 to sensors 94 94′ for detection.

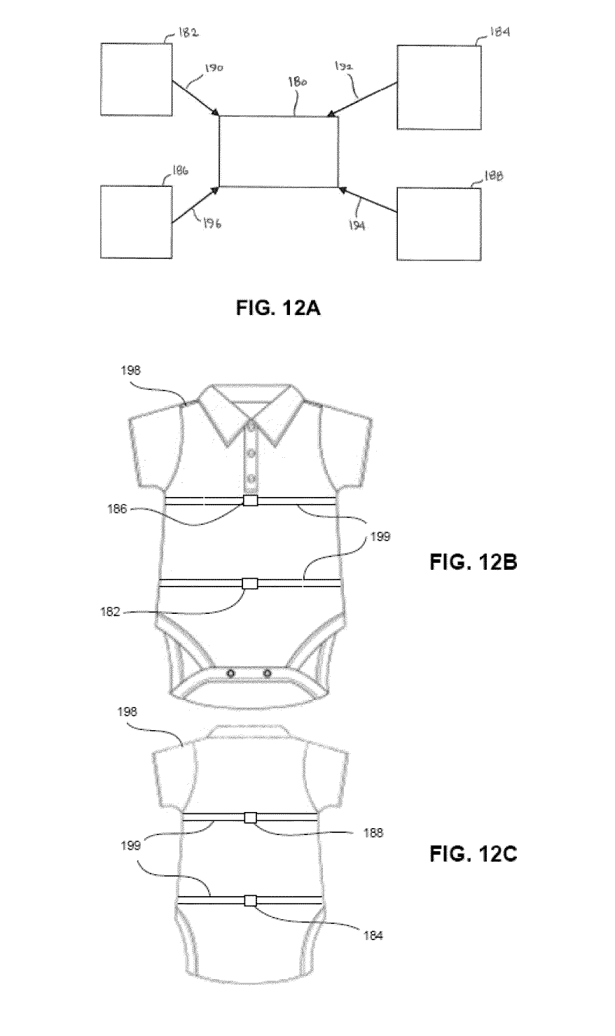

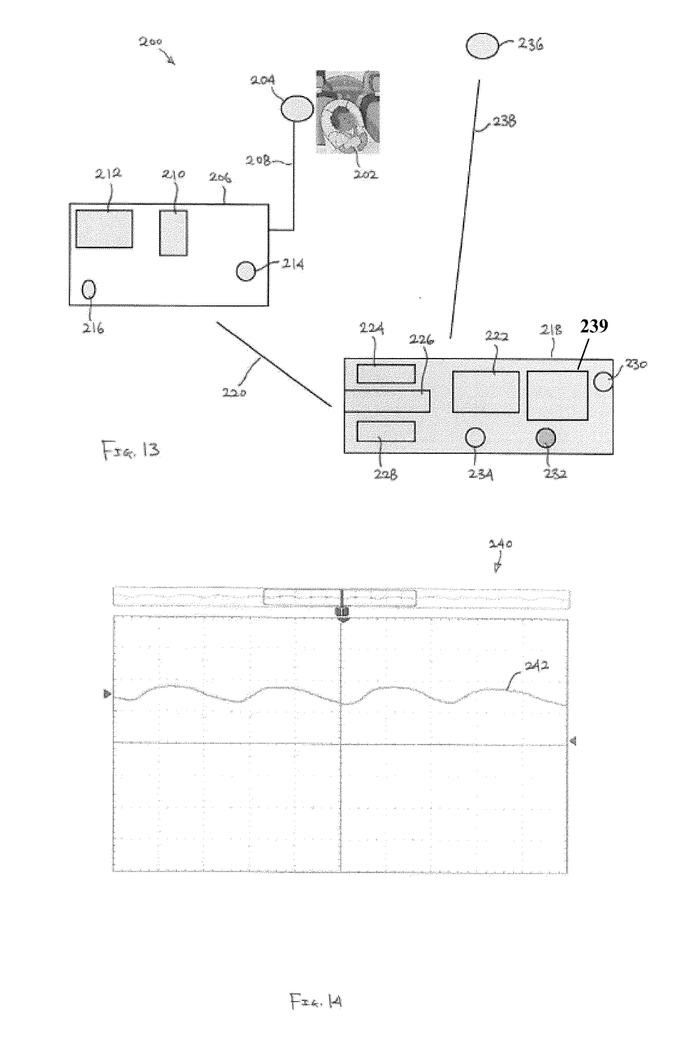

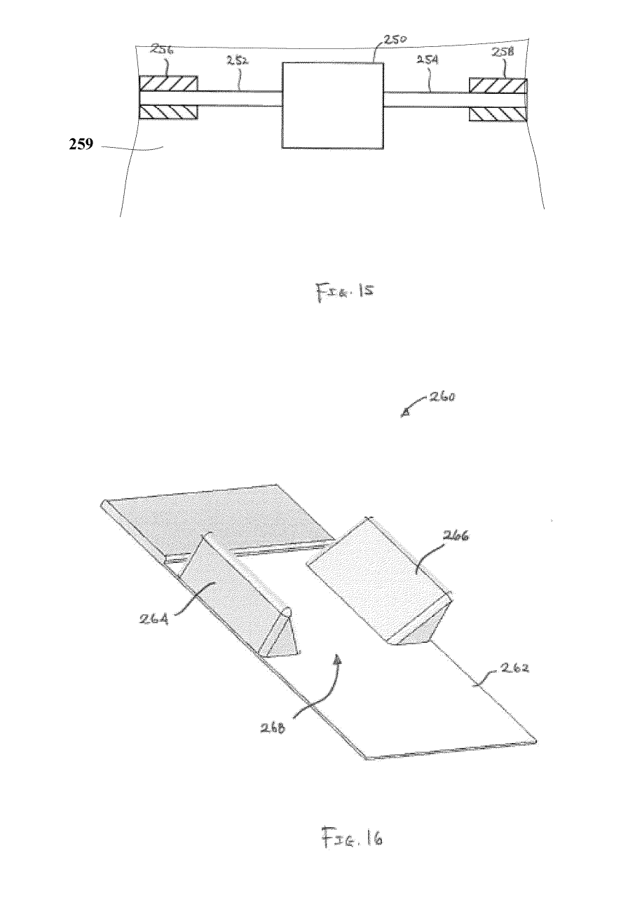

In positioning or placing the sensor modules over, upon, or in proximity to the monitored subject, a single sensor module may be positioned for instance upon the subject's chest or abdomen to detect the subject's movements associated with respiration, e.g., as the subject inhales and/or exhales, the sensor module may detect the associated movement of the chest or abdomen. Yet in other examples, multiple sensor modules may be used in combination with one another and positioned along various regions upon or in proximity to the subject's body. FIG. 12A schematically illustrates an example where a central monitoring system 180 (which may be monitored by the parent or caretaker) may be in communication with multiple sensor modules. In this example, a first module 182 may be positioned, e.g., on the belly or abdomen, a second module 184 may be positioned, e.g., on the lower back, a third module 186 may be positioned, e.g., on the chest, while a fourth module 188 may be positioned, e.g., on the upper back. Each of the sensor modules may be in communication with the central monitoring system 180 via wired transmission although wireless transmission is generally desirable to avoid wires or cables in proximity to the subject. Wireless communication is illustratively shown by the transmission communication 190, 192, 194, 196 with each respective sensor module 182, 184, 188, 186. Alternatively, individual or multiple sensor modules can be placed on multiple subjects where each of the sensor modules can communicate with the central monitoring system 180 (which may be monitored by the parent or caretaker) for each of the multiple different subjects.

FIGS. 12B and 12C illustrate the front and back, respectively, of an article of clothing such as an infant bodysuit 198, e.g., ONESIES® (Gerber Products Co., Mich.), which is typically worn by infants and how one or more of the sensor modules 182, 184, 186, 188 may be positioned upon or within. Each of the sensor modules may be integrated along the bodysuit 198 within pockets or they may be positioned via one or more optional straps 199 which are flexible and may be integrated within or along bodysuit 198 or worn separately from bodysuit 198. As previously described, although four separate sensor modules are illustrated, a single sensor module may be used and selectively positioned upon the infant or along an article of clothing worn by the infant. Alternatively, more than four sensor modules may be used over various regions of the infant, if so desired or necessary.

In addition to sensing the motion and/or breathing of the monitored subject, the monitoring system may further include a number of additional features such as audio and/or video monitoring, each of which may be used individually or in combination with one another, as shown schematically in the layout of FIG. 13. In this example, monitoring system 200 illustrates at least one sensor module 204 in contact or in proximity to the monitored subject 202, e.g., infant. The at least one sensor module 204 may be in electrical communication 208, e.g., wireless or wired, with stationary transmitter 206 which may be positioned in proximity to the monitored subject 202. Wireless transmission between the sensor module 204 and transmitter 206 may be based on the any number of wireless transmission protocols, such as ZIGBEE® (Zigbee Alliance, San Ramon, Calif.) or any other similar data transmission protocol such as BLUETOOTH® (Bluetooth Sig., Inc., Bellevue, Wash.) which allow for wireless communication in ranges up to 100 feet or more from the receiver unit 218. The stationary transmitter 206 may optionally include an audio module 212 (including, e.g., a microphone) for detecting any auditory information from subject 202, such as crying, movement, auditory signs of respiration, etc. Transmitter 206 may also include an amplifier 210 as well as an amplifier gain control 214 for adjusting the amplifier gain. A power button 216 may also be incorporated to separately power on or off the transmitter 206 if so desired while the transmitter may be powered by battery or through a standard outlet.

Transmitter 206 may further be in communication, e.g., wireless communication 220 as previously above, with a separate receiver unit 218, e.g., mobile receiver, which may be monitored by the parent and/or caretaker. Aside from the sensor module 204 and optional audio module 212, an additional video camera 236 may also be included for visually monitoring the subject 202. The video camera 236 may be in communication 238, e.g., wireless or wired, with the receiver unit 218 as well such that the parent or caretaker may also optionally visually monitor the subject 202. Wireless video communication may likewise be based on the any number of wireless transmission protocols as described above. The video camera 236 may be accordingly located in proximity to the monitored subject 202, e.g., mounted on a wall or on a crib or stand to capture video images of the subject 202. As the video camera 236 may also incorporate a wireless receiver to receive signals from the sensor module 204, transmitter module 206, and/or audio module 212, camera 236 may also include a microcontroller or video processor to integrate the sensor signal along with the video and/or audio signal as well. While the audio module 212 may detect audio signals from the subject 202, video camera 236 may also optionally include a separate audio detector, such as a microphone, to monitor any noise from the subject 202. The camera 236 may be configured to operate in both daytime and in nighttime and may operate in ranges up to 100 feet or more from the receiver unit 218 and/or monitored subject 202.

The receiver unit 218 may generally comprise a processor 222 for processing the audio and/or visual information as well as optionally processing any information relating to the subject's movement and/or breathing. Accordingly, receiver unit 218 may incorporate a respiration module 226, e.g., for detecting breathing patterns, from information received from sensor module 204, an audio module 228 for processing the optional audio signals from transmitter 206, as well as a video module 224 for processing the optional video signals from video camera 236. To view the images from video camera 236, receiver unit 218 may include a screen 239, such as an LCD screen, to display the visual images as well as the detected motion and/or breathing signals sensed by sensor module 204 either separately or simultaneously.

In one example of combining the visual signals captured by camera 236 with the information detected by sensor module 204, the system may be set to indicate the absence of any detected movement and/or breathing over a predetermined period of time. If no movement and/or breathing is detected by the sensor module 204 yet video data captured by camera 236 shows or indicates movement by the subject 202, then processor 222 in receiver unit 218 may set a false alarm code as an indication to the parent or caretaker to investigate. If movement and/or breathing is still not detected, then the alarm may increase in intensity at some predetermined time interval, e.g., 5 sec, or if the parent or caretaker fails to shut off the false alarm.

Receiver unit 218 may also include an adjustment control 234 (e.g., for adjusting volume, brightness or contrast of screen 239, etc.) as well as an adjustment control 232 for sensor module 204, e.g., for adjusting time level settings for the interval between detected movement and/or breathing episodes, for example, 10 sec, 15 sec, 20 sec, or any other minimum time period preset and/or set by the parent or caretaker. As previously described, a separate processor may be incorporated directly into sensor module 204 while processor 222 incorporated in receiver unit 218 may be utilized to optionally program the detection time interval in the sensor module 204. Processor 222 may also be programmed, as previously described, to sound a visual and/or auditory alarm to alert the parent or caretaker if the detected episodes between the subject's movement and/or breathing exceeds the programmed allowable time period. A power button 230 may also be incorporated in receiver unit 218 for powering the receiver unit 218 on or off.

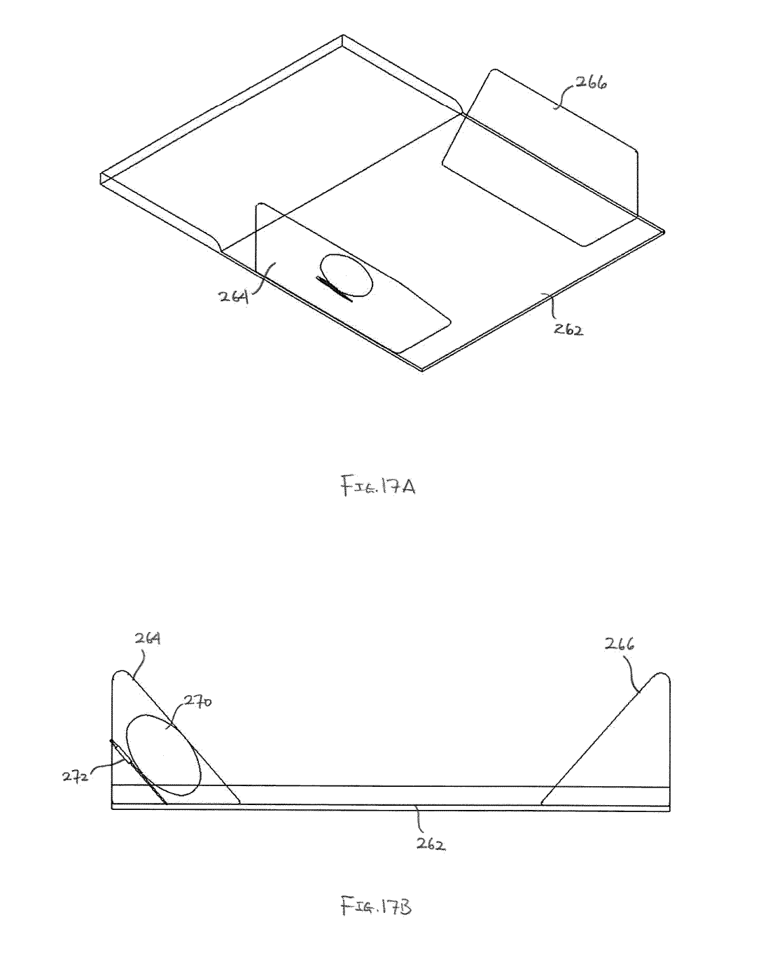

In displaying the detected motion and/or breathing signals sensed by sensor module 204 on screen 239, a waveform generator optionally incorporated in receiver unit 218 may be utilized to generate and display the detected breathing patterns and/or other waveforms illustrating normal motion/breathing states or abnormal motion/breathing states (or lack of motion/breathing), as illustrated by the exemplary detected respiration 242 in respiration graph 240 of FIG. 14. Additionally and/or alternatively, the receiver unit 218 may optionally include a wireless transmitter, e.g., Wi-Fi transmitter, to transmit any of the detected and/or processed information relating to the monitored subject 202 to the internet, particular websites, cell phones, etc. Moreover, processor 222 may also incorporate memory in receiver unit 218 to optionally record and store information relating to the subject's motion, breathing, audio and/or video information for later viewing or analysis.

In incorporating the system with respect to the monitored subject, the one or more sensor modules may be integrated into or along an article of clothing worn by the monitored subject, as previously described,. However, one or more sensor modules may be utilized in various other articles or objects such as furniture. An example is shown in the front assembly view of FIG. 15 which illustrates at least one sensor module 250 which may be positioned along, e.g., a disposable diaper 259, typically worn by a subject such as an infant. Sensor module 250 may be attached to one or more straps or belts 252, 254 (as previously described) which may each extend at a length sized to reach fasteners 256, 258 (e.g., hook and loop type fasteners) which are typically integrated on disposable diapers 259 to secure the diaper to the infant. Each of the straps or belts 252, 254 may extend to either or both of the fasteners 256, 258 such that are sized to become layered or positioned within the fasteners themselves to securely hold sensor module 250 between the fasteners 256, 258 while the diaper 259 is secured to the infant. The sensor module 250 may thus detect any movement or breathing from the subject as the abdomen moves during respiration either from the movement of the subject's body and/or from tensioning of the straps or belts 252, 254 relative to the sensor module 250, as previously described. As the diaper 259 is removed for changing, sensor module 250 and straps or belts 252, 254 may be removed and cleaned for securement to a new diaper or other article of clothing.

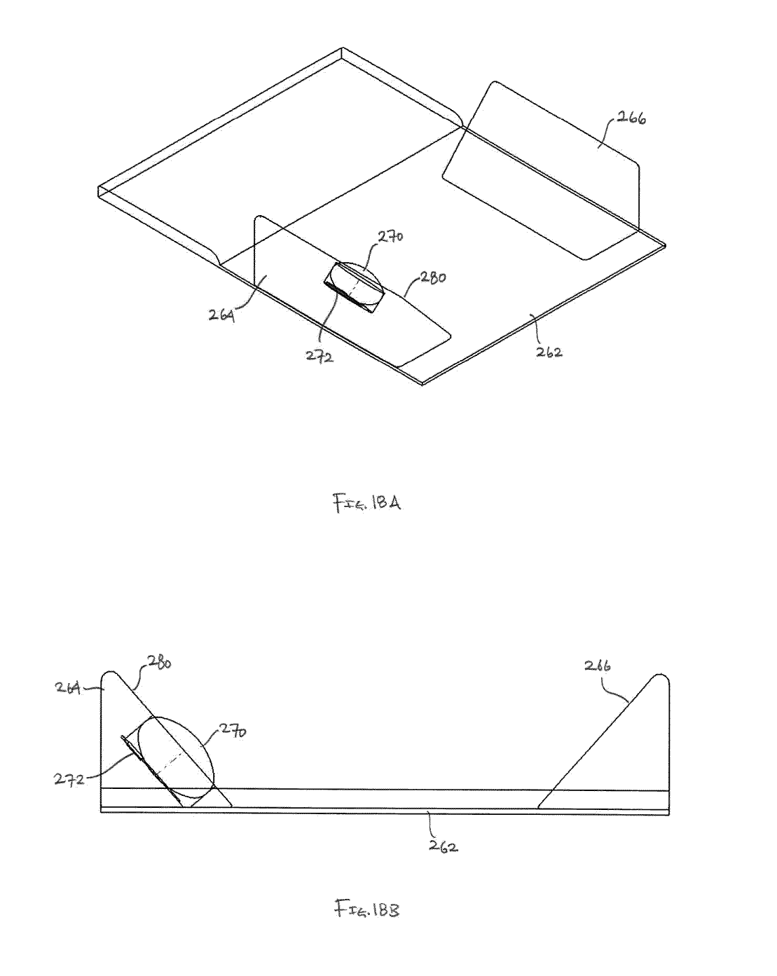

In another variation, FIG. 16 illustrates a perspective view of one example of an infant sleep positioner 260 which is typically used by parents or caretakers to prevent infants from turning over during their sleep and to keep them securely positioned. Sleep positioner 260 may typically comprise a bedding 262 having one or two adjustable protrusions or obstructions, e.g., side wedges 264, 266, which protrude from the bedding 262 to define an infant platform 268 upon which the infant may be positioned. One or more sensor modules 270 may be enclosed within one or both wedges 264, 266 such that the infant is in contact with at least one sensor module when positioned upon bedding 262. As illustrated in the respective perspective and end views of FIGS. 17A and 17B, the fluid chamber 270 of at least one sensor module may be entirely positioned, e.g., within wedge 264, such that chamber 270 comes into contact with the infant. The fluid chamber 270 may be in contact with sensor assembly 272 such that as the infant moves against wedge 264, the vibrations are transmitted through fluid chamber 270 and against sensor assembly 272. Although this and other examples illustrate a single sensor module within a single wedge 264, this is intended to be illustrative and other variations may incorporate any number of sensor modules positioned within one or both wedges 264, 266 as well as in bedding 262, as described in further detail below.

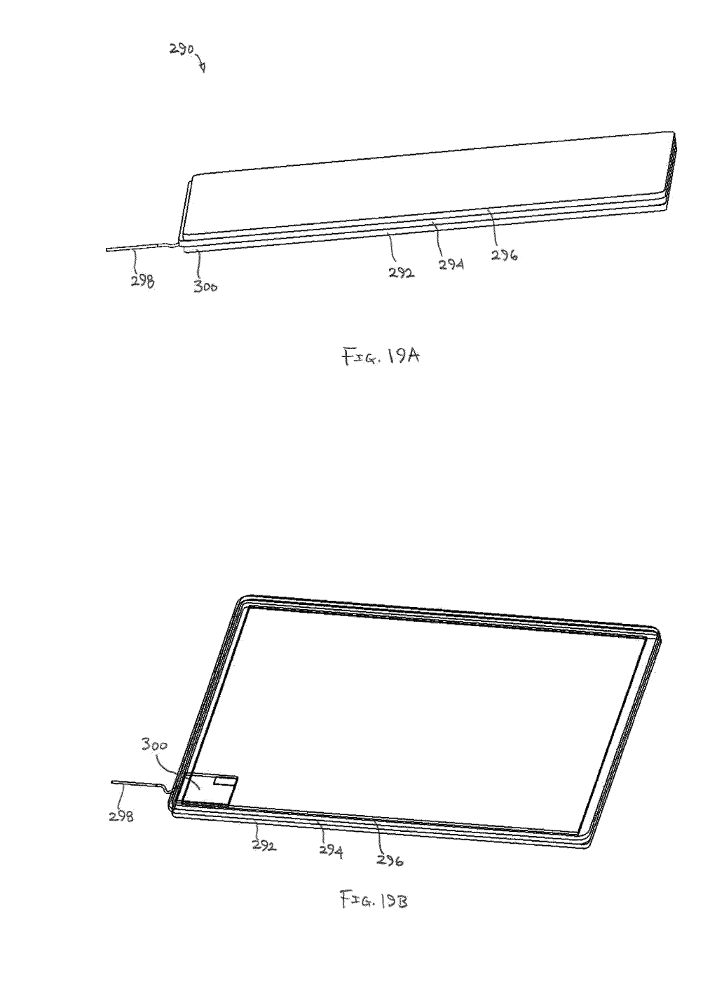

FIGS. 18A and 18B illustrate perspective and end views, respectively, of another example where at least one fluid chamber 270 of the sensor module may be positioned within or along one or both wedges 264, 266 such that fluid chamber 270 protrudes or extends beyond the wedge surface 280. When an infant is placed upon bedding 262 between wedges 264, 266, the infant may come into direct contact against the fluid chamber 270 such that movement of the infant is directly transmitted as vibrations through fluid chamber 270 and against sensor assembly 272. As above, this variation may incorporate any number of sensor modules positioned within one or both wedges 264, 266 as well as in bedding 262.

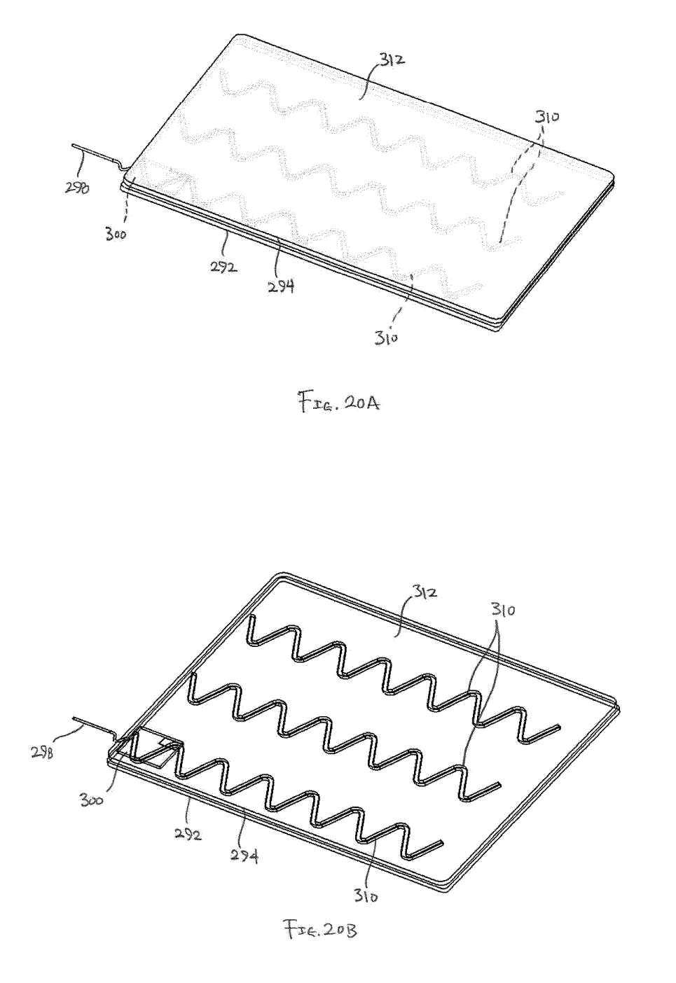

Turning now to the bedding, which may be used in combination with wedges 264, 266 or alone, an example of a bedding directly integrating a sensor assembly is illustrated in the perspective views of bedding assembly 290 in FIGS. 19A and 19B. FIG. 19A shows an assembly view of assembly 290 where a first base layer 292 may have a second fluid or gas filled layer 294 laid atop the base layer 292. A third bedding layer 296 may be laid atop the fluid or gas filled layer 294. As illustrated in the transparent perspective view of FIG. 19B, the fluid or gas filled layer 294 may transmit movement of the monitored subject through the fluid or gas as vibrations to an integrated sensor module 300, which may be integrated within base layer 292 or fluid or gas filled layer 294. The vibrations sensed by sensor module 300 may be detected and transmitted via a communication cable 298 to a receiver or other external unit, as previously described.

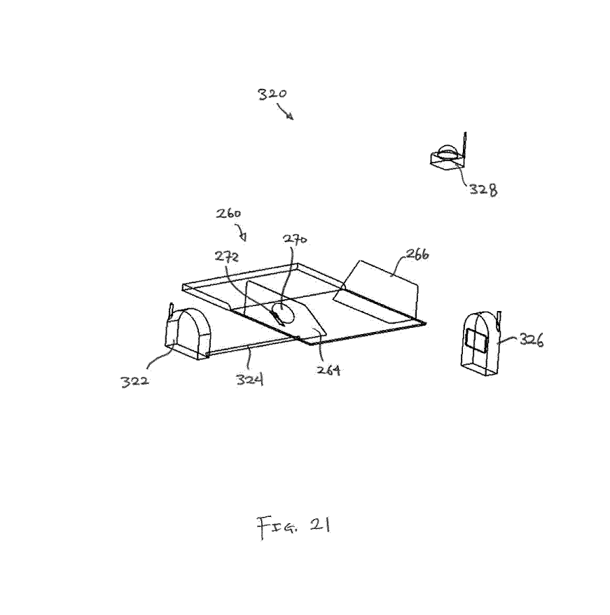

FIGS. 20A and 20B illustrate another variation where the fluid or gas filled layer 294 may incorporate the transmissive fluid or gas within a number of fluid channels 310 beneath bedding material 312. Channels 310 are shown in this example as elongate chambers which zig-zag in an alternating pattern over the entire layer 294. Each of the channels 310 may be in communication with one another and also with sensor module 300 such that movement of the monitored subject over at least one of the channels 310 may be transmitted through the length of the channels 310 and to sensor module 300 for detection. Alternative variations may have channels 310 defined in any number of configurations so long as they are in fluid communication with sensor module 300.

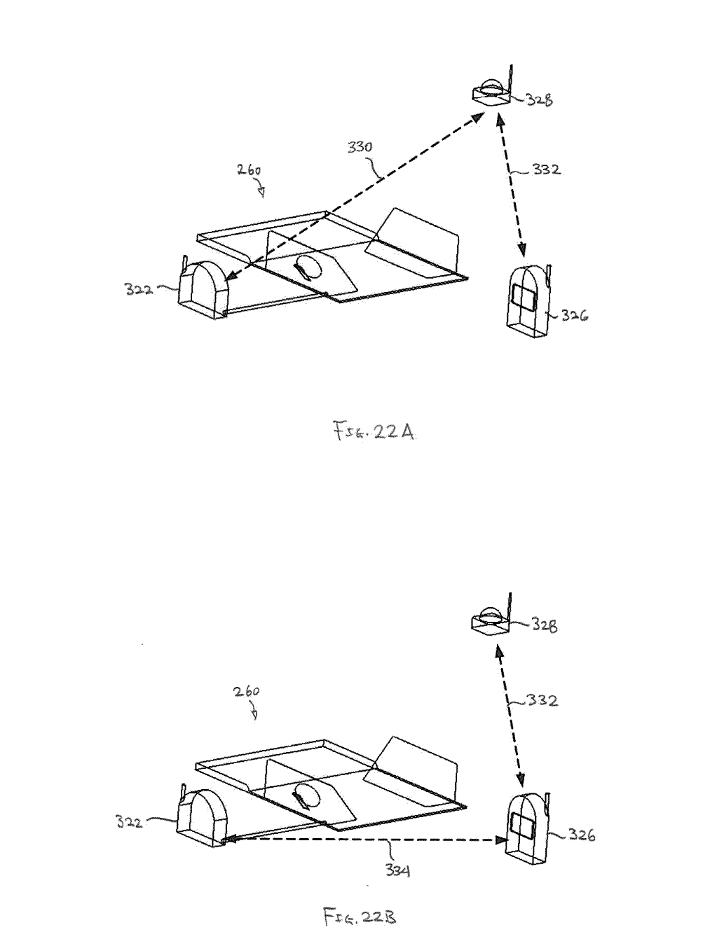

As previously described, one or more sensor modules may be used in combination with other detection features such as audio and/or video features. FIG. 21 illustrates an exemplary assembly 320 which may combine sleep positioner 260 having one or more sensor modules integrated either within one or both wedges 264, 266 and/or within the bedding. The sensor modules may be in wired or wireless communication with transmitter 322, shown in this example with cable 324, while also incorporating an optional video camera 328. Each of the transmitter 322 and/or sensor module and/or video camera 328 may be in wired or wireless communication with receiver unit 326, which is shown in this example as a portable device for use by the parent or caretaker. FIGS. 22A and 22B illustrate examples of assembly 320 and how each of the separate modules may be in communication, shown as wireless communication, with one another. For example, FIG. 22A shows how video camera 328 may be in wireless communication 330 with transmitter 322 and/or in wireless communication 332 with receiver unit 326 to allow for direct communication and data transfer therebetween. Likewise, FIG. 22B shows how receiver unit 326 may be in wireless communication 334 with either or both transmitter 322 and/or with video camera 328 to transfer data or information therebetween.

The applications of the devices and methods discussed above are not limited to the detection and/or monitoring of infants but may include any number of further detection and/or monitoring applications. Modification of the above-described device and methods for carrying out the invention, and variations of aspects of the invention that are obvious to those of skill in the art are intended to be within the scope of the claims.

What is claimed is:

1. A sensor module for detecting movement of a subject, comprising:
a housing having a rigid construction which is configured to be coupled or connected in proximity to the subject and having a contact surface defined along or within the housing, the housing further having a chamber defined therein;
a sensor which generates an electrical signal when disturbed and which is positioned within the chamber of the housing in fluid communication with the contact surface defined along the housing;
an electronics assembly integrated within or upon the housing which is in electrical communication with the sensor;
a transmissive fluid medium contained within the chamber such that the fluid medium is in fluid communication with the sensor and a vibration from the subject is received via the contact surface and transmitted through the medium and against the sensor; and,
a platform interface having a first base layer, a second fluid or gas filled layer positioned upon and separate from the first base layer, and a third layer positioned upon and separate from the second fluid or gas filled layer,
wherein the second fluid or gas filled layer defines one or more fluid channels which are in fluid communication with the sensor.

2. The module of claim 1 wherein the contact surface is comprised of a flexible material in communication with the transmissive medium.

3. The module of claim 1 wherein the housing comprises at least one connector such that tensioning of the connector induces the vibration through the medium.

4. The module of claim 1 wherein a sensitivity of the sensor is adjustable.

5. The module of claim 1 wherein the electric signal is indicative of movement by the subject.

6. The module of claim 5 further comprising a processor in electrical communication with the sensor and programmed to compare the electric signal with a nominal value in determining movement of the subject.

7. The module of claim 6 wherein the processor is programmed to indicate when successive signals are not received over a time interval.

8. The module of claim 7 wherein the time interval is adjustable.

9. The module of claim 7 wherein the time interval is adjustable over a range from 10 secs to 20 secs.

10. The module of claim 7 further comprising an alarm which indicates when the successive signals are not received over the time interval.

11. The module of claim 1 wherein the transmissive medium further comprises a gas.

12. The module of claim 1 further comprising a transmitter unit in communication with the sensor module, the transmitter unit having an audio receiver for detecting auditory information from the subject.

13. The module of claim 12 further comprising a receiver unit in communication with the transmitter unit, the receiver unit having a processor programmed to compare the electrical signal with a nominal value in determining movement of the subject.

14. The module of claim 13 wherein the receiver unit further comprises an alarm in communication with the processor.

15. The module of claim 13 further comprising a video camera in communication with the receiver unit and/or transmitter unit.

16. The module of claim 1 further comprising a sleep positioner having a bedding platform and at least one obstruction positionable relative to the bedding platform and sized to hinder movement of the subject, wherein the sensor module is positionable within or along the at least one obstruction.

17. The module of claim 1 wherein the sensor is comprised of a piezoelectric film.

18. The module of claim 1 wherein the sensor is at least partially within the housing.

19. The module of claim 1 wherein the sensor is positioned in a cantilevered configuration with respect to the housing.

20. A method of detecting movement from a subject, comprising:
providing a platform interface having a first base layer, a second fluid or gas filled layer positioned upon and separate from the first base layer, and a third layer positioned upon and separate from the second fluid or gas filled layer;
coupling or connecting a housing in proximity to the subject, where the housing has a contact surface defined along the housing, the housing further having a chamber defined therein and a sensor positioned within the housing and which generates an electrical signal when disturbed and which is contained at least partially within the housing;
transmitting a movement from the subject as a vibration through the contact surface defined along the housing and through a transmissive fluid medium contained within the chamber such that the fluid medium is in fluid communication with the sensor and vibration impinges against the sensor through the fluid medium, wherein the second fluid or gas filled layer defines one or more fluid channels which are in fluid communication with the sensor;
generating an electric signal from the sensor indicative of the movement by the subject where an electronics assembly is integrated within or upon the housing which is in electrical communication with the sensor; and,
comparing the electric signal to a predetermined nominal value in determining whether to initiate an alarm to a third party.

21. The method of claim 20 wherein positioning a housing comprises placing the housing within or along a sleep positioner in contact against the subject.

22. The method of claim 21 wherein positioning a housing comprises placing the housing within or along a bedding upon which the subject is positioned.

23. The method of claim 21 wherein transmitting a movement comprises further transmitting the vibration through a gas.

24. The method of claim 21 wherein generating an electric signal comprises adjusting a sensitivity of the sensor.

25. The method of claim 21 wherein comparing the electric signal comprises continuing monitoring of the subject if the electric signal is greater than or equal to the nominal value.

26. The method of claim 21 wherein comparing the electric signal comprises initiating the alarm if the electric signal is less than the nominal value.

27. The method of claim 21 further comprising sensing auditory information from the subject.

28. The method of claim 21 further comprising capturing visual information from the subject via a video camera.

29. The method of claim 28 further comprising integrating the visual information with information from the sensor indicative of the movement onto a visual display.

30. The method of claim 28 further comprising:
comparing the visual information for movement of the subject against information from the sensor indicative of the movement; and,
indicating a false alarm to the third party if the visual information fails to correspond to the information from the sensor.

31. The method of claim 21 further comprising transmitting information relating to the movement from the subject to a receiver unit.
